(19) United States
(12) Patent Application Publication
(10) Pub. No.: US 2025/0152965 A1
(43) Pub. Date: May 15, 2025

(54) Light-Based Beauty Device

(71) Applicant: YA-MAN LTD., Tokyo (JP)

(72) Inventor: Kazunori MAEDA, Tokyo (JP)

(21) Appl. No.: 18/918,220

(22) Filed: Oct. 17, 2024

(30) Foreign Application Priority Data
Nov. 15, 2023 (JP) 2023-194407

(51) Int. Cl. A61N 5/06; A61N 5/00

(52) U.S. Cl. CPC A61N 5/0617; A61N 2005/007; A61N 2005/0626; A61N 2005/0644; A61N 2005/0651; A61N 2005/0663

(57) ABSTRACT

A light-based beauty device is provided. The light-based beauty device includes a light emission unit, a cooling unit, and a controller. The light emission unit is configured to irradiate a skin surface with light. The cooling unit is configured to cool the skin surface by supplying airflow to the skin surface. The controller implemented by a processor is configured to allow the cooling unit to operate so that the airflow cools the skin surface irradiated with the light.

### CROSS REFERENCE TO RELATED APPLICATIONS

This application claims priority to Japanese Patent Application No. 2023-194407, filed on Nov. 15, 2023, which is expressly incorporated herein by reference in its entirety.

### BACKGROUND

### Technical Field

The present disclosure relates to a light-based beauty device.

### Related Art

A hair dryer described in U.S. Pat. No. 8,387,271 B includes a hair-growth therapeutic housing having LEDs that emit near-infrared light. U.S. Pat. No. 8,387,271 B describes that the hair dryer may blow hot air while irradiating scalp with near-infrared light.

However, when light and hot air are emitted to the scalp at the same time, the scalp becomes too hot. However, if the intensity of the light emitted by the LED is weakened in order to prevent the scalp from becoming too hot, the intensity of the light irradiating the scalp will be too weak and an adequate hair growth effect cannot be expected.

In view of the above circumstances, the present disclosure provides a light-based beauty device capable of emitting light of adequate intensity while preventing the skin surface from becoming too hot.

### SUMMARY

One aspect of the present disclosure provides a light-based beauty device. This light-based beauty device includes a light emission unit, a cooling unit, and a controller. The light emission unit is configured to irradiate a skin surface with light. The cooling unit is configured to cool the skin surface by supplying airflow to the skin surface. The controller implemented by a processor is configured to allow the cooling unit to operate so that the airflow cools the skin surface irradiated with the light.

Such an aspect allows a skin surface to be irradiated with light of adequate intensity while preventing the skin surface from becoming too hot.

### BRIEF DESCRIPTION OF THE DRAWINGS

FIG. 1 is a front perspective view of a light-based beauty device.

FIG. 2 is a rear perspective view of the light-based beauty device.

FIG. 3 is a perspective view of a state in which a light emission unit is detached from the hair dryer.

FIG. 4 is a lower perspective view of the light emission unit.

FIG. 5 is an exploded perspective view of the light emission unit.

FIG. 6A illustrates an internal configuration of a hair dryer.

FIG. 6B is an external view of the hair dryer viewed from the same angle as FIG. 6A.

FIG. 7 is a block diagram illustrating a configuration of a control circuit board of the light emission apparatus and a control circuit board of the hair dryer.

FIG. 8 is an external view illustrating an example of a portable fan operating as a cooling unit.

### DETAILED DESCRIPTION

With respect to the drawings, a description will be given of an embodiment of the present disclosure. Various features described in the following embodiments can be combined with each other.

A term “unit” in this specification may include, for example, a combination of a hardware resource implemented as circuits in a broad sense and information processing of software that can be concretely realized by the hardware resource. Furthermore, various kinds of information are described in the present embodiment, and regardless of whether such information is represented by high and low signal values as a set of binary bits consisting of 0 or 1, by physical values of signal values, or by quantum superposition, communication and computation may be executed on a circuit in a broad sense.

The circuit in a broad sense is a circuit realized by properly combining at least a circuit, circuitry, a processor, a memory, and the like. In other words, a circuit includes an application specific integrated circuit (ASIC), a programmable logic device (e.g., simple programmable logic device (SPLD)), a complex programmable logic device (CLPD), field programmable gate array (FPGA), and the like.

### Light-Based Beauty Device 1

First, a description will be given of an overall configuration of the light-based beauty device 1 of the present disclosure.

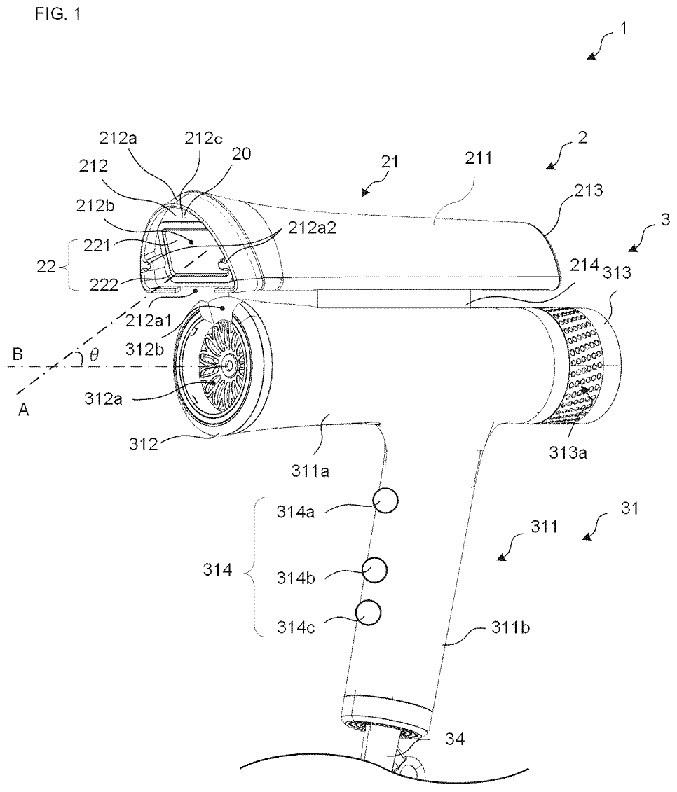

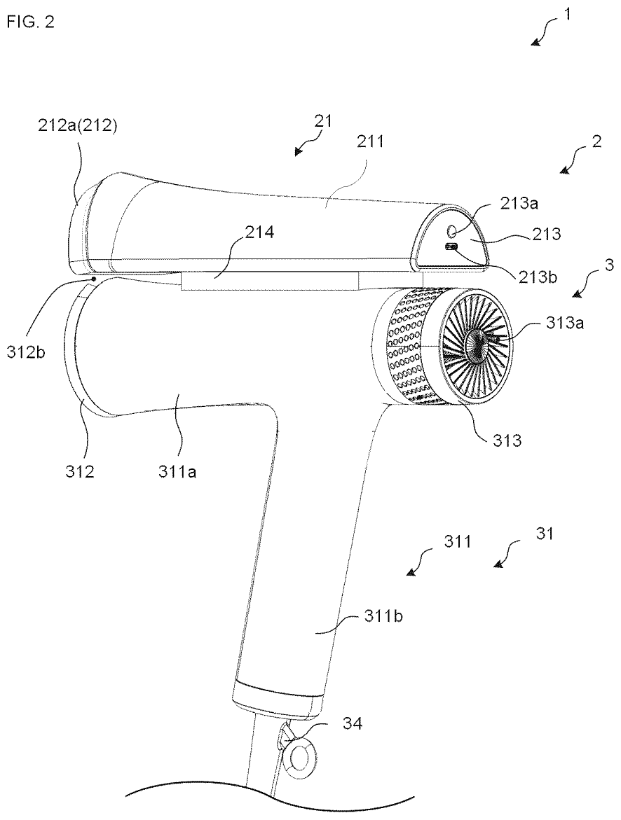

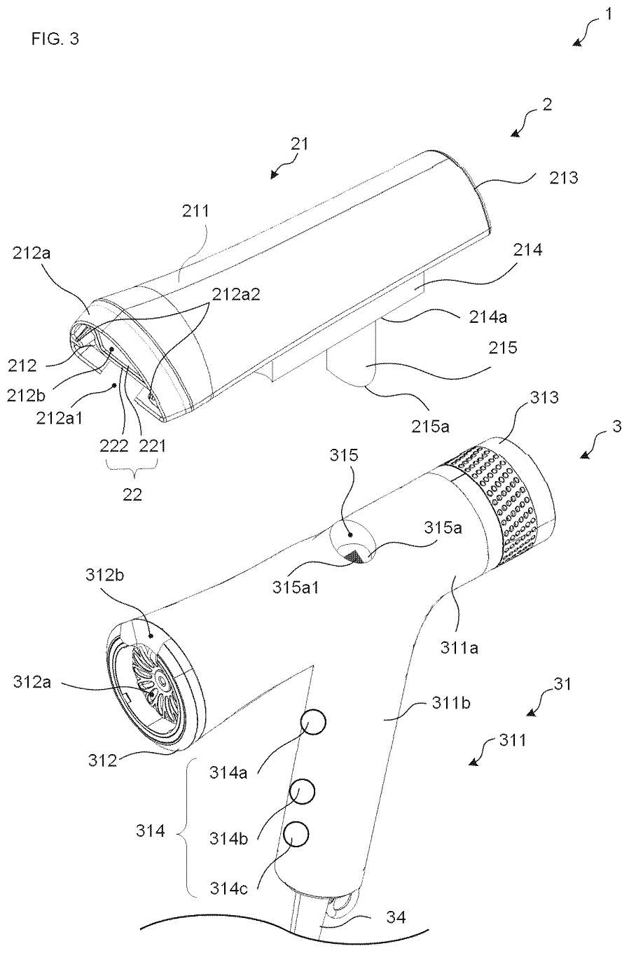

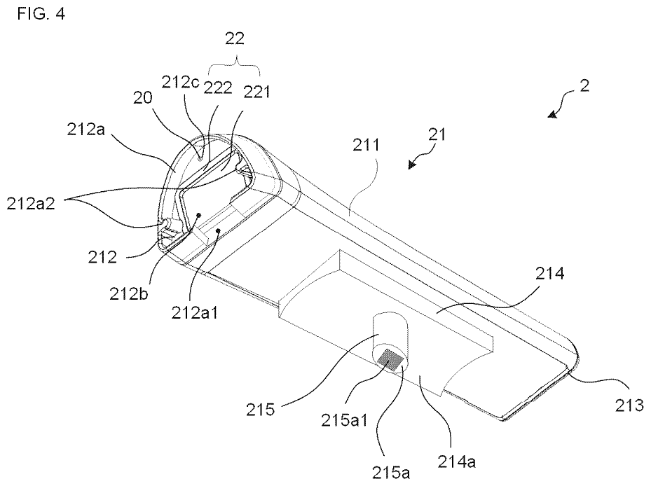

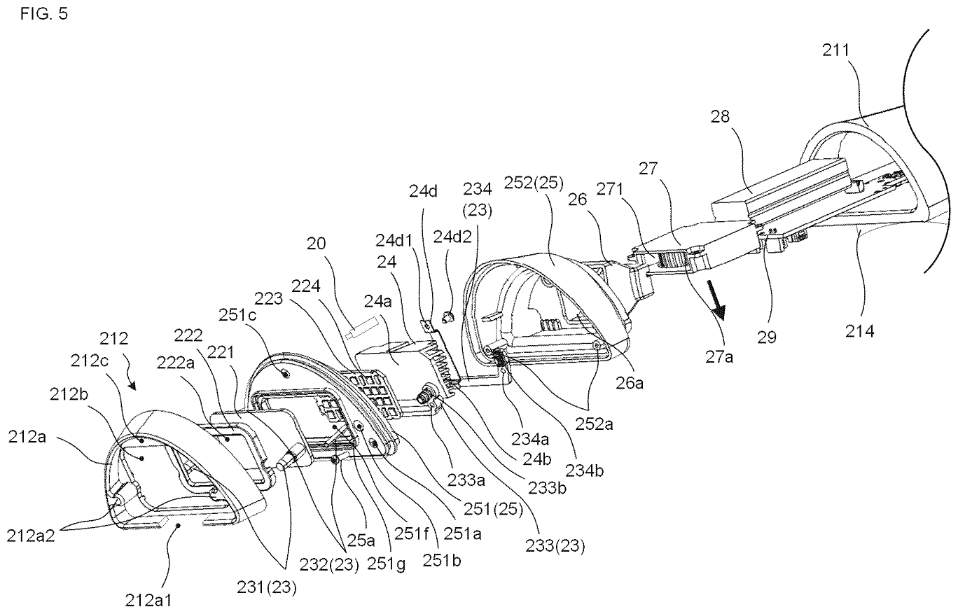

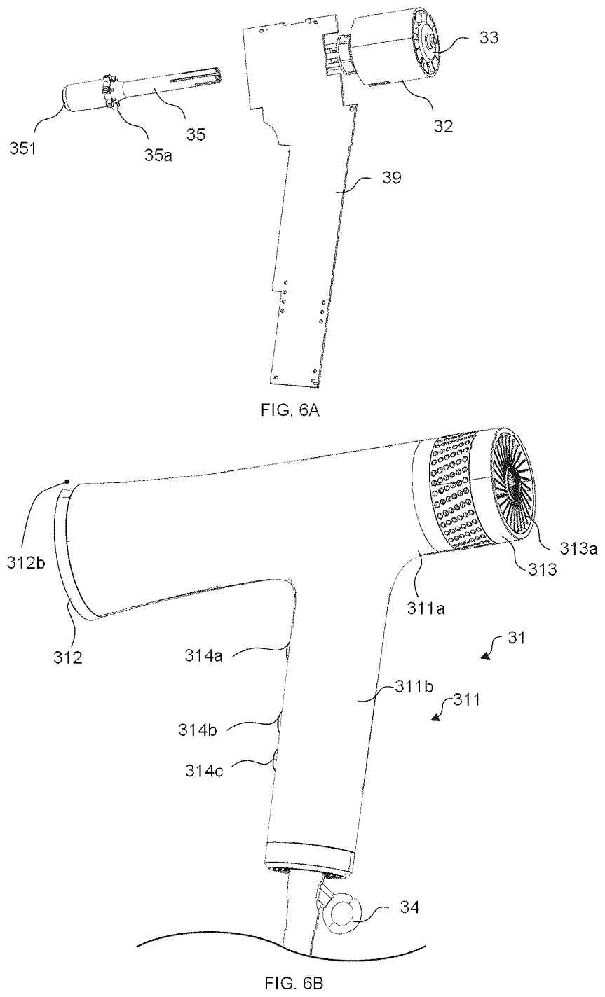

FIG. 1 is a front perspective view of a light-based beauty device. FIG. 2 is a rear perspective view of the light-based beauty device. FIG. 3 is a perspective view of a state in which a light emission unit is detached from the hair dryer. FIG. 4 is a lower perspective view of the light emission unit. FIG. 5 is an exploded perspective view of the light emission unit. FIG. 6A illustrates an internal configuration of a hair dryer. FIG. 6B is an external view of the hair dryer viewed from the same angle as FIG. 6A.

In the following description, the upper side in FIG. 1 will also be referred to as “upper”, the lower side as “lower”, the left front side as “front”, or “forward,” and the right rear side as “rear” or “back”.

The light-based beauty device 1 illustrated in FIG. 1, etc. is used on a skin surface of a user. This light-based beauty device 1 includes a light emission apparatus (light emission unit) 2 and a hair dryer (cooling unit) 3. The light emission apparatus 2 and the hair dryer 3 are detachably attachable to each other. The hair dryer (cooling unit) 3 includes an outlet port 312a through which airflow is supplied to the skin surface, and the light emission apparatus 2 is disposed at a position near (i.e., in the vicinity of or adjacent to) and above (on the lateral side of) the outlet port 312a.

The present disclosure can realize a light-based beauty device 1 with such a simple configuration. In addition, since the light-based beauty device 1 includes a hair dryer 3 and a light emission apparatus 2 detachably attachable to the hair dryer 3, the user can use the hair dryer 3 and thereafter use it as the light-based beauty device 1 by a simple operation of attaching the light emission apparatus 2 to the hair dryer 3. In other words, it is easy to make a habit of using the light-based beauty device 1.

The light emission apparatus (light emission unit) 2 is configured to emit light to a skin surface (i.e., irradiate a skin surface with light). Here, “skin surface” refers to a surface of human body, including human scalp, facial skin, body skin, hair, and the like. By appropriately setting the wavelength of the light emitted by the light emission apparatus 2, the light-based beauty device 1 can exhibit a desired beauty effect.

The hair dryer (cooling unit) 3 is configured to cool the skin surface by supplying airflow.

When light is emitted from the light emission apparatus 2, the temperature of the skin surface increases. If the temperature of the skin surface increases excessively, problems may occur such that the skin surface becomes sweaty and stuffy and hair is damaged due to protein denaturation. Many previously disclosed light-based beauty devices could not adequately lower the temperature of the skin surface or had to lower light emission intensity so as to prevent the temperature increase of the skin surface. However, the light-based beauty device 1 according to the present disclosure can cool a skin surface by airflow supplied by the hair dryer 3 while the skin surface is irradiated with light by the light emission apparatus 2. Therefore, the light-based beauty device 1 can irradiate a skin surface with high intensity light, which could not been used in the previous devices, while preventing the temperature of the skin surface from rising excessively.

In addition, there have been light-based beauty devices that reduce the temperature of a component itself that directly contacts the skin surface in order to cool the skin surface. Such a light-based beauty device may cause discomfort to the user due to excessive cooling of the skin surface.

In contrast, the light-based beauty device 1 according to the present disclosure uses airflow, thereby moderately cooling the skin surface, i.e., preventing excessive cooling of the skin surface. Hence, the user can use the light-based beauty device 1 without feeling discomfort.

As illustrated in FIGS. 3 and 4, with respect to the light-based beauty device 1, a connection leg 215 of the light emission apparatus 2 is inserted into a connection hole 315 of the hair dryer 3, and the light emission apparatus 2 is placed so that a contact face 214a of a seat 214 of the light emission apparatus 2 fits on the upper face of the hair dryer 3. This allows the light emission apparatus 2 to be attached to the hair dryer 3. The shapes for attaching the light emission apparatus 2 to the hair dryer 3 are not limited to the shapes illustrated in FIG. 3, as long as the light emission apparatus 2 can be stably attached to the hair dryer 3. For example, without the seat 214 provided, two sets of connection legs 215 and connection holes 315 may be provided side by side, front and back or left and right. For example, rather than the connection leg 215 is inserted from above into the connection hole 315, a connection leg may be slid and inserted into a connection groove extending in the front-back direction so that the light emission apparatus 2 and the hair dryer 3 may be engaged and connected, or a connection portion of the light emission apparatus 2 and a connection portion of the hair dryer 3 may be connected by magnetic force.

When using the light-based beauty device 1, the user grasps, for example, a grip cover 311b of the hair dryer 3 and brings a front end of a wall 212a into contact with the skin surface, the wall 212a being a member protruding forward of the light emission apparatus 2. By using the light-based beauty device 1 in this manner, as described in detail below, the user can accurately perform operation of allowing the hair dryer 3 to cool the skin surface during irradiation on the skin surface with light emitted from the light emission apparatus 2.

### Light Emission Apparatus 2

Next, a configuration of the light emission apparatus 2 will be described. The light emission apparatus 2 may be used together with the hair dryer 3 as a light-based beauty device 1 with an air blowing function or may be used alone as a light-based beauty device.

The light emission apparatus 2 includes an exterior member 21 and various electric components housed inside the exterior member 21. The exterior member 21 may have a sealed structure. This makes the light emission apparatus 2 waterproof and dustproof, allowing it to protect the electrical components housed inside it.

A main cover 211, a front cover 212, and a rear cover 213 of the exterior member 21 are formed from light and highly thermally conductive material such as light alloy including aluminum alloys, magnesium alloys, and titanium alloys, and copper alloy. This can make it possible to efficiently emit, to outside the light emission apparatus 2, heat generated by electric components (below-described LEDs 224, a battery 28, a control circuit board 29, etc.) housed in the exterior member 21. This makes it easy to simplify the mechanism for heat dissipation while the intensity of light emitted by the light emission apparatus 2 to the skin surface is increased i.e., while output of LED 224 increases. In other words, it is easy to provide a small-sized and lightweight light emission apparatus 2 with the beauty effect of light increased.

The main cover 211, the front cover 212, and the rear cover 213 may be made of light alloy, in particular. As a result, the light emission apparatus 2 can easily balance both mechanical strength and lightness and exhibit good waterproof performance and user-friendliness. In particular, the waterproof performance facilitates use of the light emission apparatus 2 in wet spaces such as bathrooms.

Below the main cover 211, the seat 214 and the connection leg 215 are provided. The contact face 214a of the seat 214 is a surface to be in contact with the hair dryer 3 and is curved along the upper face of the hair dryer 3. The connection leg 215 has a function of positioning the light emission apparatus 2 relative to the hair dryer 3, and is fitted into the connection hole 315 of the hair dryer 3 when the seat 214 is disposed onto the upper face of the hair dryer 3.

A bottom face 215a of the connection leg 215 has a contact 215a1.

On the rear cover 213, a power button 213a and a power hole 213b are provided as illustrated in FIG. 2. When the user presses the power button 213a, the light emission apparatus 2 is turned on. The light emission apparatus 2 can be charged when a power cord (not illustrated) is inserted into a power hole 213b. The power hole 213b has a waterproof structure (not illustrated) inside it.

The front cover 212 has an opening 212b and a wall 212a, the opening 212b being an area through which light emitted from inside the light emission apparatus 2 passes, the wall 212a surrounding the opening 212b and protruding forward. The wall 212a is disposed in such a manner that it surrounds the direction in which light passes, i.e., the wall 212a is arranged orthogonally to a light emission direction. As a result, the wall 212a is configured to be able to regulate diffusion of the light in a direction orthogonal to the light emission direction.

Such a wall 212a can allow light to be concentrated on the skin surface located in the light emission direction, which improves the effect on the skin surface to be irradiated.

Furthermore, the wall 212a can hinder light from leaking or spreading to surrounding areas other than the skin surface to be irradiated. This makes it possible to prevent light from entering the eyes, etc. of the user himself/herself or surrounding persons, and to safely use the light-based beauty device 1 or the light emission apparatus 2 even in a common space such as a washroom at home, for example.

The opening 212b is blocked by a lens 221 described below.

Specifically, the wall 212a may be configured to allow irradiation of a skin surface of an area of about 4 cm² to 20 cm², about 5 cm² to 15 cm², or about 6 cm² to 10 cm², at a time. This allows, for example, selective irradiation with light on specific areas of concern such as the parting of the hairline of the head and the hairline of the forehead. Thus, effective treatment can be expected in a short time.

On the other hand, the wall 212a may be configured to enable irradiation on a skin surface of an area of about 10 cm² or more at a time. In this case, since a wide area of the skin surface can be irradiated with light at a time, it is beneficial that irradiation can be completed in a short time when, for example, the entire scalp is to be irradiated.

The wall 212a also has a function of defining the distance from the opening (light emission unit) 212b to the skin surface by protruding forward. In other words, the wall 212a can make it possible to prevent the opening 212b from being placed too close to the skin surface, preventing the skin surface from being irradiated with excessively strong light or from becoming too hot.

At the lower part of the wall 212a, that is, on the hair dryer 3 side of the wall 212a, an indentation 212a1 is provided through which airflow supplied by the hair dryer 3 can pass. Airflow having passed through the indentation 212a1 is supplied to the inside of the wall 212a, that is, to the skin surface being irradiated with light, and thereby light irradiation and cooling by the airflow can be performed on the skin surface at the same time.

When the user uses the light-based beauty device 1 or the light emission apparatus 2, the front end of the wall 212a is brought into contact with the skin surface. The wall 212a is formed from an elastic material, and thereby the wall 212a can absorb the shock when it comes into contact with the skin surface. This can suitably reduce physical damage to the skin surface. The elastic material includes, for example, rubber materials such as silicone rubber, fluorine rubber, and natural rubber, resin materials with elasticity such as silicone resin, and the like, and in particular, the elastic material may be a medical grade silicone resin. In this case, the wall 212a can be brought into gentle contact with the skin surface. In addition, by using a medical grade silicone resin with high chemical resistance, it is easy to prevent or suppress deterioration of the wall 212a even when hair oil, lotion, etc. remaining on the skin surface adhere to the wall 212a.

The wall 212a does not have to be composed solely of an elastic material, and may be composed wholly or partially of a combination of an elastic material and a hard material (e.g., a hard resin material, a metal material, a ceramic material, etc.). For example, the wall 212a may be formed by coating a hard material with an elastic material. The wall 212a may be formed from a hard material at its rear side (opening side 212b) and from an elastic material at its front side (skin surface side). In addition, a hard portion made of a hard material may be provided on the inner or outer circumference of the wall 212a. In this case, the tip of the hard portion is positioned so that it is located behind (on the opening 212b side of) the front end of the wall 212a. The hard portion may be a wall disposed along the circumferential direction of the wall 212a, or may be a protrusion disposed on part of the circumferential direction of the wall 212a.

By the above configuration, even when the elastic portion in contact with the skin surface is contracted and deformed, the skin surface can be prevented from approaching the opening 212b (LEDs 224) beyond the hard portion.

When both of paired contact portions (contact unit) 212a2 of the wall 212a are pressed backward by an object such as a skin surface coming into contact with the contact portions 212a2, the contact of the wall 212a with the skin surface is detected by a contact detection mechanism 23 disposed inside the light emission apparatus 2. As illustrated in FIG. 1, etc., the contact portions (contact unit) 212a2 are disposed near (in the vicinity of, close to, or adjacent to) and across, in the lateral direction, the opening (light emission unit) 212b.

The number of contact portions 212a2 is not limited as long as it is two or more. For example, in addition to the pair of contact portions 212a2 on the left and right sides across the opening 212b, another pair of contact portions 212a2 may be provided on the upper and lower sides across the opening 212b.

The light emission apparatus 2 includes an intermediate accommodation unit 25 inside. The intermediate accommodation unit 25 includes an accommodation lid 251 and an accommodation body 252, and stably and compactly accommodates a temperature sensor 20, a light emission unit 22, and the contact detection mechanism 23. As illustrated in FIGS. 1 and 4, the front end part of the temperature sensor 20 is exposed to the outside through a hole 251c on the accommodation lid 251 and a hole 212c on the front cover 212. The temperature sensor 20 includes, for example, a radiation temperature sensor, and is a non-contact temperature sensor capable of measuring the temperature of the skin surface by measuring the intensity of infrared rays or visible light rays, etc. emitted from the skin surface.

The light emission unit 22 includes a support body 223 fitted into a lens frame 251g of the accommodation lid 251 from behind, two or more LEDs (light emission elements) 224 provided in an aggregated manner on the support body 223, and a lens 221 fitted into the lens frame 251g from the front, and a lens cover 222.

The accommodating lid 251 is arranged so that the two or more LEDs (light emission elements) 224 on the support body are arranged at an angle to the outlet port 312a (front cover 312) of the hair dryer 3. This makes it easier for the airflow supplied from the outlet port 312a to pass through the indentation 212a1, thereby effectively supplying both light and airflow to the skin surface. Specifically, the support body 223 may be inclined at an angle of about 3° to 45°, about 5° to 30°, or about 10° to 20° relatively to the outlet port 312a. The one-dot dashed line A in FIG. 1 is a straight line orthogonal to the support body 223, and the one-dot dashed line B is a straight line orthogonal to the outlet port 312a (front cover 312). Therefore, the angle θ between the one-dot dashed lines A and B is equal to the angle between the support body 223 and the outlet port 312a (front cover 312).

The wavelength of the light emitted by the LEDs (light emission unit) 224 may be about 600 nm to 850 nm. In other words, the light emission apparatus 2 may irradiate the skin surface with light including red light. Thus, it can be expected that, for example, effects are exhibited of promoting hair growth, promoting epidermal turnover, etc.

The wavelength of the light emitted by the LEDs (light emission unit) 224 is not limited to this, and may be longer than 490 nm and shorter than 525 nm. In other words, the light emission apparatus 2 may irradiate the skin surface with light including green light. In this case, it can be expected that, for example, an effect is exhibited of reducing melanin production.

The LEDs (light emission unit) 224 may be configured to irradiate the skin surface with red light and green light simultaneously or alternately, or may be configured to irradiate the skin surface with light of other wavelengths having a beauty effect.

The intensity of the light emitted by the light emission units (LEDs 224) 2 may be, on the skin surface, about 6 mW/cm² or more, about 100 mW/cm² or more, about 150 mW/cm² or more, about 200 mW/cm² or more, about 250 mW/cm² or more, or about 300 mW/cm² or more. This allows light of adequate intensity to be given to cells in the hair follicles, dermis, etc., and thereby, for example, effects can be expected of promoting hair growth, improving skin conditions, etc. When the intensity of the light emitted by the LEDs 224 is about 250 mW/cm² to 300 mW/cm², it is easy achieve both safety and beauty effect.

A fluorescent lamp, HID lamp, krypton lamp, xenon lamp, laser element, etc., for example, may be provided on the support body 223 instead of or together with the two or more LEDs 224. The light emission apparatus 2 may not include two or more light sources, but may have a single light source as long as light of the necessary intensity can be provided to the skin surface.

The light emitted by the LEDs 224 passes through the lens 221 provided in the front direction of the LED 224. The lens 221 is fitted into the lens frame 251g together with the support body 223, and is fixed to the lens frame 251g and the front cover 212 by a lens cover 222. The lens cover 222 is in close contact with the outer circumference of the lens frame 251g and the inner circumference of the opening 212b and thereby prevents the lens 221 from falling off and liquid from entering inside the light emission apparatus 2.

The positions of the lens 221, the opening 251f, the opening 222a of the lens cover 222, and the opening 212b are aligned, and thereby light having passed through the lens 221 passes the opening 212b to the outside of the light emission apparatus 2 and irradiates the skin surface.

The contact detection mechanism 23 includes a pair of waterproof cones 231, a pair of touch pins 232, a sensor cover 233 having a pair of extension-retraction protrusions 233b, and a touch sensor 234 having a pair of detection portions 234b.

When the contact portion 212a2 of the wall 212a is pressed backward, the waterproof cone 231 located inside (behind) the contact portion 212a2 is also pressed.

Each waterproof cone 231 is fitted tightly into a hole 251b of the accommodation lid 251, which optimally prevents water from entering the inside of the light emission apparatus 2. The waterproof cone 231 expands and contracts, thereby moving backward the touch pin 232 positioned inside. The touch pin 232 having moved backward presses the extension-retraction protrusion 233b of the sensor cover 233 backward.

The sensor cover 233 is configured to reliably prevent water, dust, etc. from reaching the touch sensor 234. The extension-retraction protrusion 233b is configured to transmit the pressure force from the touch pin 232 to the detection portion 234b of the touch sensor 234 by retracting backward when the touch pin 232 presses it.

The number of each of waterproof cone 231, touch pin 232, extension-retraction protrusion 233b, and detection portion 234b is not limited to two, and may be suitably set based on the number of contact portions.

The light emission unit 22 and the contact detection mechanism 23 are fixed to the intermediate accommodation unit 25 with screws 25a. Each screw 25a is fastened in a hole 252a of the accommodation body 252 through a hole 251a of the accommodation lid 251, a hole 233a of the sensor cover 233, and a hole 234a of the touch sensor 234.

Behind the support body 223 of the light emission unit 22, a heat sink 24 is disposed. The heat sink 24 is fixed to the accommodation lid 251 by a mounting plate 24d. Specifically, a screw 24d2 is fastened in a hole (not illustrated) of the accommodation lid 251 through a hole 24d1 of the mounting plate 24d.

A heat-receiving surface 24a of the heat sink 24 is closely in contact with the back of the support body, and thereby receives heat generated by the LEDs 224 via the support body 223. This heat is transferred to heat radiation fins 24b provided on the rear side of the heat sink 24, and from the heat radiation fins 24b to the air in an air vent 26a of a duct 26 provided behind the heat sink 24.

The light emission apparatus 2 further includes a battery 28, a control circuit board 29, and a fan 27 inside it. The battery 28 is configured to store electric power supplied from a utility power source, etc. via the power hole 213b on the rear cover 213 and to supply electric power to the entire light emission apparatus 2. The control circuit board 29 controls the operation of the entire light emission apparatus 2 and is configured to, via the contact 215a1, detect the connection of the hair dryer 3 or communicate with the hair dryer 3.

The fan 27 has a function of collecting the heat generated by the battery 28 and the control circuit board 29 and the heat of the air in the air vent 26a. Specifically, blades 271 of the fan 27 rotate to intake air from the front and rear directions into the air vent 27a and exhaust the air in the direction of the arrow (downward) in FIG. 5. The heat contained in the exhausted air is absorbed by the main cover 211, which has high thermal conductivity as described above, and is quickly discharged to the outside. Hence, the light emission apparatus 2 does not require heat exhaust holes and can thus have an excellent waterproof and dustproof configuration.

### Hair Dryer 3

Next, a description will be given of the hair dryer 3.

The hair dryer 3 can be used as the light-based beauty device 1 together with the light emission apparatus 2 or used alone as an air blow apparatus. When the user simply wants to dry the hair, the user can remove the light emission apparatus 2 and use the hair dryer 3 in a lightweight condition.

The hair dryer 3 includes an exterior member 31 and various electric components accommodated inside the exterior member 31. The exterior member 31 includes a main cover 311, a front cover 312, and a rear cover 313.

The rear cover 313 includes, as illustrated in FIG. 2, an inlet port 313a for take outside air into the hair dryer 3. As illustrated in FIG. 1, the front cover 312 includes an outlet port 312a for supplying airflow from inside the hair dryer 3 to the outside. The inlet port 313a and outlet port 312a are not limited to the shapes illustrated in the drawings, as long as they can exhibit the above-described functions.

The front cover 312 has a shape surrounding the outlet port 312a and protrudes forward beyond the outlet port 312a. This prevents the airflow supplied from the outlet port 312a from spreading out and facilitates to shape the airflow into a straight line, thereby enabling rapid drying of the hair, etc.

On the other hand, on the upper side of the front cover 312, i.e., the side to which the light emission apparatus 2 is attached, an indentation 312b is provided through which airflow supplied from the outlet port 312a can pass. The airflow that passing through the indentation 312b passes through the indentation 212a1 of the wall 212a of the light emission apparatus 2 and is supplied to inside the wall 212a. This allows the airflow to be supplied to and cool the skin surface inside the wall 212a, i.e., the skin surface being irradiated with light. Thus, the indentation 312b and the indentation 212a1 form the passage through which adequate airflow can be supplied inside the wall 212a. Such a light-based beauty device 1 can adequately lower the temperature of the skin surface to, for example, 53° C. or lower or 44° C. or lower, preventing excessive increase in the temperature of the skin surface even when the skin surface is irradiated with strong light. Therefore, a high beauty effect can be expected without skin problems caused by the temperature increase on the skin surface.

The main cover 311 consists of an air channel cover 311a and a grip cover 311b. The air channel cover 311a forms an air channel through which the airflow taken in from the inlet port 313a is supplied from the outlet port 312a.

The upper face of the air channel cover 311a has a connection hole 315 into which the connection leg 215 of the light emission apparatus 2 is inserted. The hole bottom surface 315a of the connection hole 315 has a contact 315a1 configured to, by contacting the contact 215a1 of the connection leg 215, detect the attachment of the light emission apparatus 2 or communicate with the light emission apparatus 2. The hair dryer 3 may further includes a lid configured to be mated with the connection hole 315. The lid is configured to be attachable to the connection hole 315 when the light emission apparatus 2 is not attached to (is removed from) the hair dryer 3. This prevents foreign matters from entering inside the hair dryer 3 (connection hole 315).

The grip cover 311b is a part that can be gripped by the user. An interface unit 314 is disposed on the front face of the grip cover 311b.

The interface unit 314 includes a power button 314a, a hot air button 314b, and a cool air button 314c, which are provided at positions easily pressed by the user. The power button 314a can turn on the power of the hair dryer 3 by being pressed. The hot air button 314b and the cool air button 314c are buttons for, when pressed, changing the airflow supplied from the outlet port 312a to hot air and room temperature air (i.e., cool air), respectively. The configurations of the interface unit 314 are not limited to this, and in addition to or instead of the buttons 314a, 314b, or 314c, the interface unit 314 may have other buttons such as the flow rate change button that allows the flow rate to be changed.

A power cord 34 is provided at the lower end of the grip cover 311b to supply power to the hair dryer 3 from a utility power source or the like.

As illustrated in FIG. 6, the hair dryer 3 includes, inside, a motor 32, a control circuit board 39, and a heater 35.

The motor 32 generates airflow by rotating the impeller 33 installed behind the motor and thereby taking outside air from the inlet port 313a of the rear cover 313. The heater 35 is configured to heat the airflow generated by the motor 32 and passing through the air channel cover 311a. The heater 35 is connected to a connection portion 351 via a fitting portion 35a, and the connection portion 351 is fixed to the outlet port 312a of the front cover 312. The hot air, which is airflow heated by the heater 35, or airflow that is not heated and remains at a room temperature is supplied to the skin surface through the outlet port 312a.

The control circuit board 39 controls the operation of the entire hair dryer 3 and is configured to, via the contact 315a1, detect the attachment of the light emission apparatus 2 or communicate with the light emission apparatus 2.

### Controller

Next, a description will be given of control executed in the light-based beauty device 1. The control circuit board 29 and the control circuit board 39 function as a controller of the light-based beauty device 1.

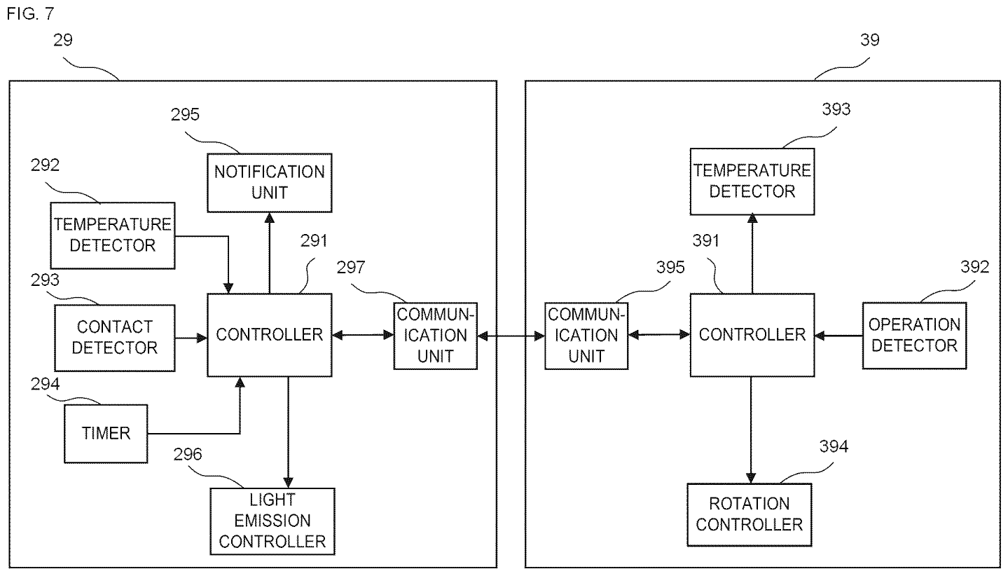

FIG. 7 is a block diagram illustrating a configuration of the control circuit board of the light emission apparatus and the control circuit board of the hair dryer.

The control circuit board 29 of the light emission apparatus 2 includes a controller 291, a temperature detector 292, a contact detector 293, a timer 294, a notification unit 295, a light emission controller 296, and a communication unit 297.

The controller 291 executes processing and control on the overall operation pertaining to the light emission apparatus 2. The controller 291 is, for example, a processing circuit (not illustrated). The temperature detector 292 detects the temperature of the skin surface measured by the temperature sensor 20. The contact detector (contact determination unit) 293 determines whether or not the pair of contact portions (contact unit) 212a2 contacts the skin surface (object). The timer 294 measures an elapsed time since the LED 224 started emitting light. The notification unit 295, for example, vibrates a diaphragm (not illustrated) to generate a buzzing sound. The notification unit 295 is not particularly limited as long as it issues a notification to the user, and may, for example, cause light emitted by the LEDs 224 to blink, the light irradiation by the LEDs 224 to stop, vibration by vibrating a vibrator, or the like. The light emission controller 296 executes control on the LEDs 224, such as light emission/stop, intensity adjustment, etc. of the LEDs 224. The communication unit (attachment-detachment detector) 297 is configured to detect, via the contact 215a1, the attachment or detachment (i.e., removal) of the hair dryer 3.

Based on the detected temperature of the skin surface communicated from the temperature detector 292, the controller 291 allows at least one of an emission intensity, an emission pattern, or wavelength of light emitted by the LEDs (light emission unit) to be changed. The controller 291 may have at least one of them changed so that the detected temperature is 53° C. or less or 44° C. or less. This can more reliably prevent or hinder the user from feeling pain or getting burnt due to excessive heating of the skin surface.

For example, when the temperature of the skin surface exceeds 53° C., the controller 291 allows the light emission controller 296 to control power supply to the LEDs 224 and thereby to lower the emission intensity, make the emission pattern intermittent, and lengthen the wavelength of the light emitted from the LEDs 224. Alternatively, when the temperature of the skin surface is above 44° C. but 53° C. or less, the controller 291 allows the light emission controller 296 to control power supply to the LEDs 224 and thereby to make the emission pattern intermittent and not to change the emission intensity and the wavelength of the light emitted by the LEDs 224. These combinations of temperature and control are mere examples, and at least one of the emission intensity, emission pattern, or wavelength of the light emitted by the LEDs 224 may be changed so that the temperature of the skin surface lowers.

Conversely, when the detected temperature of the skin surface communicated from by the temperature detector 292 is lower than 53° C. or 44° C., the controller 291 may control the light emission controller 296 so that at least one of an increase in the light emission intensity, a change from intermittent emission to continuous emission, or a change in the wavelength to a shorter wavelength is executed.

Such a configuration can reduce the light amount irradiating the skin surface when the temperature of the skin surface rises. Therefore, skin problems due to excessive increase in the temperature of the skin surface can be more effectively prevented. Conversely, when the temperature of the skin surface has not increased, the effect of hair growth, etc. can be increased by increasing the light amount. In other words, while both overheating of the skin surface and inadequate light intensity are prevented, light of high intensity can be supplied to the skin surface, and thus a high effect can be expected even with a short use time.

The controller 291 is configured to issue a notification to the user of the light-based beauty device 1 in response to communication from the timer 294 indicating that a predetermined time has elapsed since the LEDs (light emission unit) 224 started emitting light. Specifically, when a predetermined time has elapsed since the LEDs 224 started emitting light, the controller 291 allows the notification unit 295 to issue a notification to the user.

The predetermined time may be set, for example, to about 2 to 10 seconds, about 3 to 8 seconds, or about 4 to 6 seconds. The predetermined time may not be fixed. For example, based on the emission intensity of the light emitted by the LEDs 224, a time may be calculated such that light of adequate intensity can be applied to the skin surface, and the calculated time may be set as the predetermined time.

The notification unit 295 notifies the user of the elapse of the predetermined time, enabling the user to be aware that the skin surface has been irradiated with necessary and adequate light. Then, the user can stop the light emission to the skin surface without concerns about inadequate irradiation and can start light emission to the next position of the skin surface that the user wishes to irradiate with light. In addition, this can prevent prolonged continuous exposure to light on a specific part of the skin surface, allowing the user to use the light-based beauty device 1 without concerns about skin problems, etc. caused by irradiation for a long period of time. Furthermore, the time required to use the light-based beauty device 1 is short, further facilitating making a habit of the daily use of the light-based beauty device 1.

The controller 291 further receives a notification from the contact detector 293 as to whether or not the contact portion 212a2 contacted an object. When both of the paired detection portions 234b of the touch sensor 234 are adequately pressed, that is, when it is determined that both (all) of the paired contact portions (two or more contact portions) 212a2 contact the skin surface (object), the contact detector 293 determines that the pair of contact portions (contact unit) 212a2 contacts the object.

When a notification of “no” is communicated from the contact detector 293, the controller 291 allows the light emission controller 296 to stop the light emission from the LEDs 224. In other words, when the contact detector (contact determination unit) 293 determines that the pair of contact portions (contact unit) 212a2 does not contact an object, the controller 291 prohibits the LED (light emission unit) 224 from emitting light (does not allow the LED (light emission unit) 224 to emit light).

This can prevent the LEDs 224 from emitting light when, for example, the wall 212a does not contact with the skin surface, or when the wall 212a is in contact with the skin surface at an angle and therefore only one of the paired contact portions 212a2 is being pressed. Thus, when the wall 212a is not in adequate contact with the skin surface, the LEDs 224 does not emit light, suitably avoiding any danger such as danger of light entering the eyes of the user or the like.

The controller 291 receives a notification from the communication unit 297 as to whether or not the hair dryer 3 is attached. When the controller (light emission unit) 291 receives a notification from the communication unit 297 indicating (i.e., detects by the attachment-detachment detector) that the hair dryer (cooling unit) 297 is not attached, the function of the LEDs (light emission unit) 224 is restricted. Specifically, the controller 291 controls the light emission controller 296 to reduce the irradiation intensity of the light emitted by the LEDs (light emission unit) 224, to change the light emission pattern to intermittent irradiation, or to lengthen the wavelength. The controller 291 may control the light emission controller 296 to make all these changes, or any one or two of them.

This can reduce the intensity of light supplied to the skin surface when the hair dryer 3 is not attached, that is, when the airflow cannot be supplied to the skin surface. Therefore, even when the hair dryer 3 is not attached, the temperature of the skin surface can be suitably prevented or hindered from rising too high.

The communication unit 297 may be configured not only to detect whether or not the hair dryer 3 is attached, but also to transmit, to the hair dryer 3, information obtained by the light emission apparatus 2, such as the temperature of the skin surface, the presence or absence of contact between the skin surface and the contact portions 212a2, the elapsed time since the LEDs 224 started emitting light, or the like.

The communication unit 297 may be configured to receive information from the hair dryer 3. For example, the communication unit 297 may receive whether or not the hair dryer 3 is supplying airflow and notify the controller 291 of it. The controller 291 may be configured to stop or restrict light emission by the LEDs (light emission unit) 224 when the hair dryer (cooling unit) 3 is not supplying airflow. Such a configuration can prevent or hinder the skin surface from being irradiated with strong light when the skin surface is not cooled by airflow. Therefore, the temperature of the skin surface can be suitably prevented from rising too much.

The control circuit board 39 of the hair dryer 3 includes a controller 391, an operation detector 392, a temperature controller 393, a rotation controller 394, and a communication unit 395.

The controller 391 executes processing and controls on the overall operation pertaining to the hair dryer 3. The controller 391 is, for example, a processing circuit (not illustrated). The operation detector 392 detects whether or not any of the interface unit 314 has been operated. The temperature controller 393 controls the output to the heater 35 to adjust the temperature of the airflow. The rotation controller 394 controls the rotation of the motor 32 to adjust the flow rate (air amount) of the airflow. The communication unit (attachment-detachment detector) 395 is configured to detect, via the contact 315a1, the attachment/detachment of the light emission apparatus 2.

The controller 391 controls the temperature controller 393 based on the operation on the interface unit 314 communicated from the operation detector 392.

For example, in response to a notification indicating that the hot air button 314b is pressed, the temperature controller 393 executes a control such that the heater 35 operates and thereby hot air is supplied from the outlet port 312a. In response to a notification indicating that the cool air button 314c is pressed, the temperature controller 393 executes a control such that the heater 35 stops, and thereby the airflow at room temperature is supplied from the outlet port 312a. The hair dryer 3 may also include a cooling element such as a Peltier element and may be configured to provide an electric current to the cooling element to cool the airflow when the cool air button is pressed.

In a case where the interface unit 314 includes a flow rate change button, in response to a notification indicating that the flow rate change button is pressed, the controller 394 allows the rotation controller to increase or decrease the rotation speed of the motor 32 to increase or decrease the flow rate (air amount) of the airflow supplied from the outlet port 312a. For example, the flow rate of the airflow may be changed in three levels: about 1 m/s or higher, about 5 m/s or higher, and about 10 m/s or higher.

In response to a notification from the communication unit 395 indicating that the light emission apparatus 2 is attached and that the light emission apparatus 2 is operating, the controller 391 allows the temperature controller 393 to stop the heater 35 and the rotation controller 394 to rotate the motor 32. In other words, the controller 391 allows the hair dryer (cooling unit) 3 to operate so that the airflow cools the skin surface irradiated with light from the light emission apparatus (light emission apparatus) 2. This prevents hot air from being supplied to the skin surface being irradiated with light and ensures airflow supply to the skin surface during light irradiation. This can prevent or hinder an excessive increase in the temperature of the skin surface.

In this case, the airflow supplied to the skin surface may be continuous, intermittent, or a combination thereof.

The communication unit 395 may further be configured to receive from the light emission apparatus 2 whether or not light emission starts and to notify the controller 391 of it. In this case, the controller 391 does not allow the temperature controller 393 to stop the heater 35 when light emission does not start even in a case where the light emission apparatus 2 is attached. Then, when the light irradiation starts, control is provided to the temperature controller 393 to stop the heater 35. As a result, the hair dryer 3 can be used as a normal hair dryer with the light emission apparatus 2 is attached, and the light-based beauty device 1 can cool the skin surface by airflow without fail when it is used as a light-based beauty device 1.

By executing the above-described control, the light-based beauty device 1 can irradiate the skin surface with adequate light while preventing the skin surface from becoming too hot.

The following describes modifications of the above-described light-based beauty device 1. The above embodiment and the following modifications can be combined with each other in any combination.

In the above-described embodiment, the light-based beauty device 1 determines whether or not the pair of contact portions (contact unit) 212a2 contacts the skin surface (object). However, the light-based beauty device 1 may be configured to determine a distance between the skin surface (object) and the LEDs 224 instead of or in addition to the determination of the contact. When the skin surface is strongly pressed against the contact portions 212a2, the wall 212a, which is formed from elastic material, is deformed and the distance between the skin surface and the LEDs 224 decreases.

The light-based beauty device 1 may determine the distance between the skin surface and the LEDs 224 based on the strength of the pressure on the contact portions 212a2. Alternatively, the light-based beauty device 1 may include a separate contact sensor or a distance measuring sensor, such as a laser, electrostatic, or LED-light sensor, and use it to determine the distance.

The light-based beauty device 1 may be configured to determine the position of the skin surface in order of distance from the LEDs 224, i.e., from the front. The light-based beauty device 1 may, for example, be configured to detect a first measurement distance and a second measurement distance. Here, the first measurement distance refers to a distance between the skin surface and the LEDs 224 when the skin surface having approached from the front is in close contact with the front edge of the wall 212a and therefore the light emitted by the light emission apparatus 2 does not leak from between the skin surface and the wall 212a. Specifically, when the distance between the skin surface and the LEDs 224 is the first measurement distance, the skin surface is located about 5 mm behind the front edge of the wall 212a in a natural state (i.e., a state where no external force is applied to the wall 212a). In other words, the first measurement distance is a distance at which light leakage to the surroundings can be prevented and the airflow supplied to between the lens 221 and the skin surface can suitably flow (or circulate) for the intensity of the light emitted to the skin surface.

On the other hand, the second measurement distance refers to a distance between the skin surface and the LEDs 224 at which the space between the skin surface and the lens 221 (i.e., the space inside the wall 212a) is not sufficient. Specifically, when the distance between the skin surface and the LEDs 224 is the second measurement distance, the skin surface is located about 5 mm in front of the lens 221, for example. In other words, the second measurement distance is a distance such that the airflow cannot adequately cool the skin surface due to inadequate flow (or circulation) of the airflow.

In other words, the arrangement and settings of each part of the light-based beauty device 1 are, in order from the front, the front end of the wall 212a, the position of the skin surface when the first measurement distance is determined, the position of the skin surface when the second measurement distance is determined, the lens 221, and the outlet port 312a.

When the light-based beauty device 1 determines that the distance between the skin surface and the LEDs 224 exceeds the first measurement distance, the light-based beauty device 1 may prohibit the LEDs 224 from emitting light. When the light-based beauty device 1 determines that the distance between the skin surface and the LEDs 224 is less than or equal to the first measurement distance and greater than or equal to the second measurement distance, the light-based beauty device 1 may permit the LEDs 224 to emit light. Furthermore, when the light-based beauty device 1 determines that the distance between the skin surface and the LEDs 224 is the second measurement distance or less, the light-based beauty device 1 may prohibit or reduce the light emission from the LEDs 224, may change the light emission pattern or wavelength, or may increase or cool the airflow supplied from the outlet port 312a.

The control executed by the light-based beauty device 1 is not limited to the above examples. In other words, the light-based beauty device 1 may execute control such that when the skin surface is in close contact with the wall 212a to the extent that light does not leak into the surrounding area, light emission from the LEDs 224 is permitted, and that when the distance between the skin surface and the LEDs 224 is too close and the skin surface becomes unnecessarily hot, the skin surface is not irradiated with too strong light or the degree of cooling of the skin surface is increased.

As described above, determining the distance between the skin surface and the LEDs 224 step by step and controlling the light emission or the airflow supply based on the determination result can further improve the safety of the light-based beauty device 1.

The light-based beauty device 1 may be configured to detect further one or more measurement distances. In this case, as the distance between the skin surface and the LEDs 224 decreases, a stepwise control may be executed of, for example, decreasing the intensity of the light emitted from the LEDs 224, changing the irradiation pattern (light emission pattern) to shorten the irradiation time, changing the emitted light to a longer wavelength, increasing the flow rate of the airflow supplied from the outlet port 312a, or the like. The control is not limited to those. This can further improve the safety of the light-based beauty device 1.

The control based on the detection result of the measured distance may be executed by either the controller 291 or the controller 391, or by both.

In the above-described embodiment, the light emission apparatus 2 includes the temperature sensor 20, but instead of the light emission apparatus 2, the hair dryer 3 may include a temperature sensor. In this case, the detected temperature of the skin surface is communicated to the controller 391. Then, the controller 391 is configured to allow at least one of the flow rate, temperature, or flow direction of the airflow supplied by the hair dryer (cooling unit) 3 to be changed based on the detected temperature of the skin surface. Specifically, for example, when the temperature of the skin surface exceeds 44° C. or 53° C., the controller 391 is configured to change at least one of the flow rate, temperature, or flow direction of the airflow.

When the flow rate of the airflow is to be changed, the rotation controller 394 may increase the rotation speed of the motor 32 to increase the flow rate (air amount) of the airflow. When the temperature of the airflow is to be changed, the temperature controller 393 may allow the heater 35 to stop or, in a case where the hair dryer 3 has a cooling element such as a Peltier element, activate the cooling element to cool the airflow. When the airflow direction is to be changed, for example, the hair dryer 3 may be configured to change the angle of the outlet port 312a in the hair dryer 3 and may change the angle of the outlet port 312a to supply the airflow toward the upper direction (i.e., toward the skin surface irradiated with light).

In addition, each of the light emission apparatus 2 and the hair dryer 3 may include a temperature sensor. In this case, when the temperature of the skin surface rises, each of the light emission apparatus 2 and the hair dryer 3 can execute a control to cool the skin surface. Therefore, the overheating of the skin surface can be prevented or hindered more efficiently.

In addition, a configuration may be such that a temperature sensor is provided in one of the light emission apparatus 2 or the hair dryer 3, and the temperature is transmitted to the other via the communication unit 297 and the communication unit 395.

In the above-described embodiment, each of the light emission apparatus 2 and the hair dryer 3 includes a controller. However, either one of the light emission apparatus (light emission unit) 2 or the hair dryer (cooling unit) 3 may include a controller.

In this case, one of them may execute the function as the controller of the other and be configured to allow each part of the other to operate via the communication unit 297 and the communication unit 395.

In the above-described embodiment, the hair dryer 3 operates as a cooling unit, but the light emission apparatus 2 may also include a cooling unit. For example, the front cover 212 of the light emission apparatus 2 may have an outlet port, and a fan may be provided inside the light emission apparatus 2. Thereby, both the light emission apparatus 2 and the hair dryer 3, or the light emission apparatus 2 alone, may be configured to supply airflow to the skin surface.

In the above-described embodiment, each of the light emission apparatus 2 and the hair dryer 3 is configured to detect each other's attachment state (whether or not the other is attached) by using the contact 215a1 and the contact 315a1. However, how to detect the attachment state is not particularly limited. For example, they may notify each other of their attachment state via wireless communication or by detecting a change in magnetic force.

In addition, either one of the light emission apparatus 2 or the hair dryer 3 may be configured to detect the attachment state of the other. For example, when only the hair dryer 3 is capable of detecting the attachment state of the light emission apparatus 2, the hair dryer 3 may detect the attachment state of the light emission apparatus 2 by having a switch that is pressed when the light emission apparatus 2 is attached, or by detecting the weight of the light emission apparatus 2.

In the above-described embodiment, the light emission apparatus 2 is located on the upper side of the outlet port 312a of the hair dryer 3, but the light emission apparatus 2 may be positioned, for example, on the side in the lateral direction of or below the outlet port 312a.

It is to be noted, when the light emission apparatus 2 is positioned on the upper side of the hair dryer 3, it is possible to easily configure or omit a mechanism to prevent the light emission apparatus 2 from falling off or the like.

In the above-described embodiment, the light emission apparatus 2 is waterproof and dustproof, but the light emission apparatus 2 need not be waterproof and dustproof. In this case, the fan 27 may not be a sealed fan, and a heat exhaust hole may be provided on the main cover 211 of the light emission apparatus 2. In addition, the exterior member 21 may be formed from a material with low thermal conductivity such as resin.

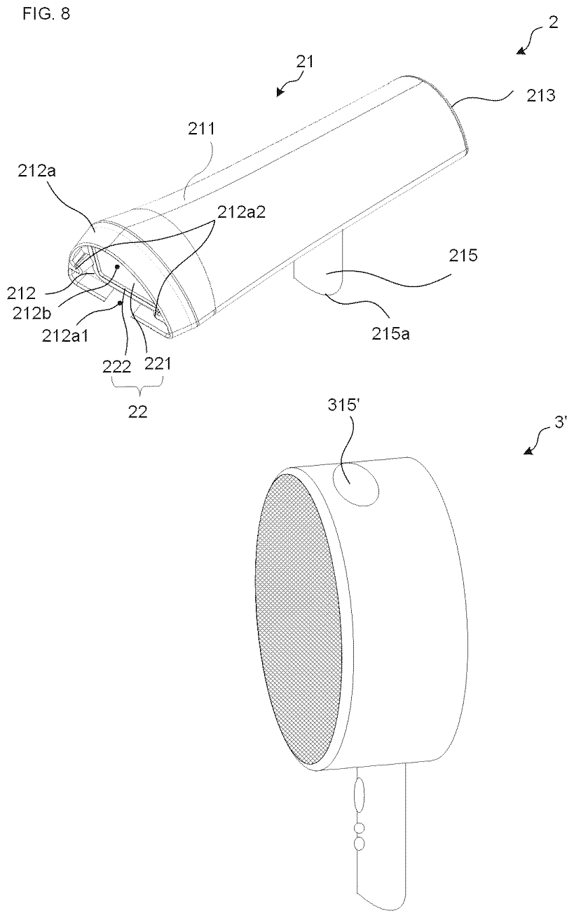

In the above-described embodiment, the hair dryer 3 operates as a cooling unit, but the cooling unit is not limited to a hair dryer. FIG. 8 is an external view illustrating an example where a portable fan operates as a cooling unit.

The upper part of the portable fan 3′ has a connection hole 315′ allowing insertion of the connection leg 215 of the light emission apparatus 2. The connection hole 315′ may have a contact (not illustrated) at the bottom.

The cooling unit does not have to be a hair dryer 3 or a portable fan 3′, but may be an air blower, body dryer, or the like.

The present disclosure may be provided in each of the following aspects.

(1) A light-based beauty device comprising: a light emission unit configured to emit light to a skin surface; a cooling unit configured to cool the skin surface by supplying airflow to the skin surface; and a controller configured to allow the cooling unit to operate so that the airflow cools the skin surface irradiated with the light.

(2) The light-based beauty device according to (1), wherein intensity of the light emitted by the light emission unit is 6 mW/cm² or more on the skin surface.

(3) The light-based beauty device according to (1) or (2), wherein a wavelength of the light emitted by the light emission unit includes a range from 600 nm to 850 nm.

(4) The light-based beauty device according to any one of (1) to (3), further comprising a temperature detector configured to detect a temperature of the skin surface.

(5) The light-based beauty device according to (4), wherein based on the detected temperature of the skin surface, the controller allows at least one of emission intensity, an emission pattern, or a wavelength of the light emitted by the light emission unit to be changed.

(6) The light-based beauty device according to (4) or (5), wherein based on the detected temperature, the controller allows at least one of a flow rate, a temperature, or a flow direction of the airflow supplied by the cooling unit to be changed.

(7) The light-based beauty device according to (6), wherein the controller allows the at least one to be changed so that the detected temperature is 44° C. or less.

(8) The light-based beauty device according to any one of (1) to (7), wherein when the cooling unit is not supplying the airflow, the controller stops or restricts emission of the light by the light emission unit.

(9) The light-based beauty device according to any one of (1) to (8), wherein when a predetermined time has elapsed since the light emission unit started emitting the light, the controller issues a notification to a user of the light-based beauty device.

(10) The light-based beauty device according to any one of (1) to (9), further comprising: a contact unit disposed near the light emission unit; and a contact determination unit configured to determine whether or not the contact unit contacts an object.

(11) The light-based beauty device according to (10), wherein the contact unit has two or more contact portions, and when the contact determination unit determines that all of the contact portions contact the object, the contact determination unit determines that the contact unit contacts the object.

(12) The light-based beauty device according to (10) or (11), wherein when the contact determination unit determines that the contact unit does not contact the object, the controller does not allow the light emission unit to emit the light.

(13) The light-based beauty device according to any one of (1) to (12), wherein the light emission unit further includes a wall configured to regulate diffusion of the light in a direction orthogonal to an emission direction of the light.

(14) The light-based beauty device according to (13), wherein the wall includes an indentation through which the airflow can pass, the wall being configured to define a distance from the light emission unit to the skin surface.

(15) The light-based beauty device according to (13) or (14), wherein the wall is formed from elastic material.

(16) The light-based beauty device according to any one of (1) to (15), wherein the cooling unit includes an outlet port configured to allow the airflow to pass through it and be supplied to the skin surface, and the light emission unit includes two or more light emission elements arranged in an aggregate manner, the light emission elements being disposed at a position near and on a lateral side of the outlet port.

(17) The light-based beauty device according to (16), wherein the light emission elements are arranged at an angle to the outlet port.

(18) The light-based beauty device according to (1), wherein the light emission unit and the cooling unit are detachably attachable to each other, and at least one of the light emission unit or the cooling unit includes the controller.

(19) The light-based beauty device according to (18), wherein at least one of the light emission unit or the cooling unit includes an attachment-detachment detector configured to detect attachment or detachment of the other.

(20) The light-based beauty device according to (19), wherein when the attachment-detachment detector detects that the cooling unit is not attached, function of the light emission unit is restricted.

(21) The light-based beauty device according to any one of (1) to (20), the cooling unit is a hair dryer.

Of course, the present disclosure is not limited to these.

As described above, various embodiments of the present disclosure have been described, but these are presented as mere examples and are note intended to limit the scope of the disclosure. Novel embodiments can be implemented in various other forms, and various omissions, replacements, and modifications can be made within the scope of the spirit of the disclosure. The embodiments and its modifications are included in the scope and the spirit of the disclosure and are included in the scope of the invention described in claims and the equivalent scope thereof.

For example, the light emission apparatus 2 and the hair dryer 3 may not be detachably attachable to each other, but may be integrally configured as a whole.

What is claimed is:

1. A light-based beauty device comprising:
a light emission unit configured to emit light to a skin surface;
a cooling unit configured to cool the skin surface by supplying airflow to the skin surface; and
a controller implemented by a processor configured to allow the cooling unit to operate so that the airflow cools the skin surface irradiated with the light.

2. The light-based beauty device according to claim 1, wherein
intensity of the light emitted by the light emission unit is 6 mW/cm² or more on the skin surface.

3. The light-based beauty device according to claim 1, wherein
a wavelength of the light emitted by the light emission unit includes a range from 600 nm to 850 nm.

4. The light-based beauty device according to claim 1, further comprising a temperature detector configured to detect a temperature of the skin surface.

5. The light-based beauty device according to claim 4, wherein
based on the detected temperature of the skin surface, the controller allows at least one of emission intensity, an emission pattern, or a wavelength of the light emitted by the light emission unit to be changed.

6. The light-based beauty device according to claim 4, wherein
based on the detected temperature, the controller allows at least one of a flow rate, a temperature, or a flow direction of the airflow supplied by the cooling unit to be changed.

7. The light-based beauty device according to claim 6, wherein
the controller allows the at least one to be changed so that the detected temperature is 44° C. or less.

8. The light-based beauty device according to claim 1, wherein
when the cooling unit is not supplying the airflow, the controller stops or restricts emission of the light by the light emission unit.

9. The light-based beauty device according to claim 1, wherein
when a predetermined time has elapsed since the light emission unit started emitting the light, the controller issues a notification to a user of the light-based beauty device.

10. The light-based beauty device according to claim 1, further comprising:
a contact unit disposed near the light emission unit; and
a contact determination unit configured to determine whether or not the contact unit contacts an object.

11. The light-based beauty device according to claim 10, wherein
the contact unit has two or more contact portions, and
when the contact determination unit determines that all of the contact portions contact the object, the contact determination unit determines that the contact unit contacts the object.

12. The light-based beauty device according to claim 10, wherein
when the contact determination unit determines that the contact unit does not contact the object, the controller does not allow the light emission unit to emit the light.

13. The light-based beauty device according to claim 1, wherein
the light emission unit further includes a wall configured to regulate diffusion of the light in a direction orthogonal to an emission direction of the light.

14. The light-based beauty device according to claim 13, wherein
the wall includes an indentation through which the airflow can pass, the wall being configured to define a distance from the light emission unit to the skin surface.

15. The light-based beauty device according to claim 13, wherein
the wall is formed from elastic material.

16. The light-based beauty device according to claim 1, wherein
the cooling unit includes an outlet port configured to allow the airflow to pass through it and be supplied to the skin surface, and
the light emission unit includes two or more light emission elements arranged in an aggregate manner, the light emission elements being disposed at a position near and on a lateral side of the outlet port.

17. The light-based beauty device according to claim 16, wherein
the light emission elements are arranged at an angle to the outlet port.

18. The light-based beauty device according to claim 1, wherein
the light emission unit and the cooling unit are detachably attachable to each other, and
at least one of the light emission unit or the cooling unit includes the controller.

19. The light-based beauty device according to claim 18, wherein
at least one of the light emission unit or the cooling unit includes an attachment-detachment detector configured to detect attachment or detachment of the other, and
when the attachment-detachment detector detects that the cooling unit is not attached, function of the light emission unit is restricted.

20. The light-based beauty device according to claim 1, wherein
the cooling unit is a hair dryer.
